(19) United States
(12) Patent Application Publication
(10) Pub. No.: US 2010/0279861 A1
(43) Pub. Date: Nov. 4, 2010

(54) Antifungal Agents

(76) Inventors: Gerald F Bills, Madrid (ES); Javier Collado, Madrid (ES); Craig A. Parish, Tenafly, NJ (US); Fernando Pelaez, Madrid (ES); Gonzalo Platas, Madrid (ES); Terry Roemer, Montreal (CA)

Correspondence Address: MERCK, P O BOX 2000, RAHWAY, NJ, 07065-0907, US

(21) Appl. No.: 12/680,835

(22) PCT Filed: Aug. 12, 2008
(86) PCT No.: PCT/US08/09610

§ 371 Date: Mar. 30, 2010

Related U.S. Application Data
(60) Provisional application No. 60/999,110, filed on Oct. 16, 2007.

(30) Foreign Application Priority Data
Aug. 16, 2007 (ES) 200702289

(51) Int. Cl. A01N 43/40; C12P 17/18; C12N 1/14; A61K 38/02; A61K 38/12; A61K 31/7048; A61K 31/513; A61K 31/4745; C07D 498/16; A01N 43/80; A61P 31/04; A01P 3/00; A01P 5/00; A01P 7/04; A01P 13/00; A01P 15/00

(52) U.S. Cl. 504/101; 435/119; 435/254.1; 514/2.2; 514/3.6; 514/31; 514/274; 514/279; 546/41; 504/130

(57) ABSTRACT

Novel isoxazolidinone compounds useful in the treatment and/or prevention of human and animal fungal infections, as well as in the control of phytopathogenic fungi in crops are described. Two novel strains of fungi, ATCC No. PAT-7894 and ATCC No. PAT-7895, are disclosed for the preparation of the described isoxazolidinone compounds.

### FIELD OF THE INVENTION

The present invention relates to isoxazolidinone compounds that are derived by fermentation and that are useful as antifungal agents.

### BACKGROUND OF THE INVENTION

U.S. Pat. No. 6,017,924 describes pyridonoquinolines, pyridonopyrrolidinoquinolines and related compounds useful as androgen receptor modulators. In addition, WO 97/49709 describes non-steroidal aza polycycles that are useful as androgen receptor modulators.

Structures and activities of many ergochrome secondary fungal metabolites, including secalonic acids, are known. Von B. Franck & H. Flasch, Die Ergochrome (Physiologie, Isolierung, Strucker and Biosynthie), 30 Fortschritte der Chemie Organischer Naturstoffe 151-206 (1973) describe structures and properties of ergochrome AA (secalonic acid A), ergochrome BB (secalonic acid B), ergochrome AB (secalonic acid C) and ergochrome EE (secalonic acid D). Colin C. Howard & Robert A. W. Johnstone, Fungal Metabolites. Part IV. Crystal and Molecular Structure of Secalonic Acid A, J.C.S. Perkins Transactions 11820-1822 (1976) describe crystal and molecular structure of secalonic acid A. Raymond Anderson et al., Secalonic Acids D and F Are Toxic Metabolites of Aspergillus aculeatus, 42(2) J. Org. Chem. 352-353 (1977) describe secalonic acids D and F as toxic metabolites of Aspergillus aculeatus. Itsuo Kurobane et al., A New Secalonic Acid, Linkage Between Tetrahydroxanthone Units Determined From Deuterium Isotope ¹³C Chemical Shifts, Tet. Lett. 4633-4636 (1978) describe the structure of secalonic acid G, isolated from Pyrenochaeta terrestris. Istuo Kurobane et al., Biosynthetic Relationships Among the Secalonic Acids. Isolation of Emodin, Endocrocin and Secalonic Acids from Pyrenochaeta terrestris and Aspergillus aculeatus, 32(12) J. Antibiot. 1256-1266 (1979) describe isolation of emodin, endocrocin and secalonic acids A, E and G from Pyrenochaeta terrestris, isolation of emodin, endocrocin and secalonic acids B, D and F from Aspergillus aculeatus, and biosynthetic relationships among the secalonic acids. A. E. Pohland et al., Physicochemical Data for Some Selected Mycotoxins, 54(11) Pure & Appl. Chem. 2219-2284 (1982) describe physicochemical data for selected mycotoxins, including secalonic acid D. Charles L. Barnes et al., Crystal Structures of Methanol and Ethanol Solvates of Secalonic Acid D, 29(9) J. Chem. Crystallography 1031-1035 (1999) describe crystal structures of methanol and ethanol solvates of secalonic acid D. George R. Pettit et al., Isolation and Structure of Ruprechstyril from Ruprechtia tangarana, 66 J. Nat. Prod. 1065-1069 (2003) describe isolation and structure of ruprechstyril from Ruprechtia tangarana. Richard J. Cole & Richard H. Cox, Handbook of Toxic Fungal Metabolites, 647-661 (Academic Press 1981) describe chemical and toxicity data for secalonic acids A, B, C and D.

However, there remains a need for potent antifungal agents for general use against pathogens associated with human and agricultural fungal infections.

### SUMMARY OF THE INVENTION

The present invention relates to compounds that are selected from the group consisting of compounds of formulas I and II

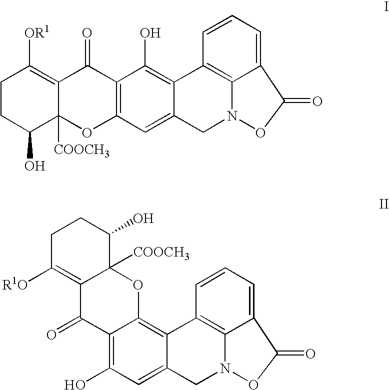

or pharmaceutically or agriculturally acceptable salts thereof. In formulas I and II, R¹ is selected from the group consisting of hydrogen and C₁-C₆ alkyl groups, and R¹ is hydrogen in particular embodiments. These compounds are potent antifungal agents with broad spectra of activity and can be used against pathogens associated with human and agricultural fungal infections.

Additional aspects of the invention relate to compositions comprising mixtures of the compounds of the invention and pharmaceutical and agricultural compositions and formulations that comprise a compound of the invention. In addition, aspects of the invention relate to methods of preparing a compound of the invention, to methods of treating or preventing fungal infection in humans and animals using a compound of the invention, and to methods of controlling fungal infection in humans, animals and plant materials using a compound of the invention.

### DETAILED DESCRIPTION OF THE INVENTION

One aspect of the present invention provides a purified compound selected from the group consisting of compounds of formulas I and II, pharmaceutically acceptable salts thereof, and agriculturally acceptable salts thereof. In formulas I and II, R¹ is selected from the group consisting of hydrogen and C₁-C₆ alkyl groups. In particular embodiments, R¹ is hydrogen.

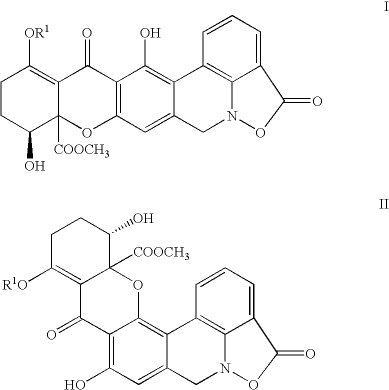

The compounds of the invention may be obtained from biological samples, as described below, may be produced by chemical modification of compounds obtained from biological samples, or may be synthesized chemically. The compounds of the invention may be provided as naturally occurring compounds and mixtures of compounds, or may be isolated and purified to produce “purified” compounds and/or compositions.

As used herein, the term “purified” refers to compounds or compositions provided in a form that is substantially lacking any components other than the desired compounds of formula I or II and their salts; for example, a composition comprising a mixture of compounds of formulas I and II and their salts, in which R¹ is hydrogen, may be referred to as a “purified” composition if provided in a form substantially lacking in any fungal components other than the claimed mixture.

Another aspect of the invention provides a composition, which comprises one or more compounds selected from the group consisting of compounds of formula I or II and pharmaceutically or agriculturally acceptable salts thereof. In embodiments, such a composition may be purified. In addition, the composition may be racemic mixtures of such compounds, in embodiments.

Another aspect of the invention provides a pharmaceutical composition, which comprises one or more compounds of formula I or II or a salt thereof and a pharmaceutically acceptable carrier.

Suitable pharmaceutically acceptable salts of the compounds of formulas I and II include, for example, inorganic base salts, such as alkali metal salts (e.g., sodium and potassium salts), ammonium salts, and organic base salts. Suitable organic base salts include amine salts, such as tetra-alkyl-ammonium salts (e.g. tetrabutylammonium and trimethylcetylammonium), trialkylamine salts (e.g. triethylamine), dialkyl amine salts (dicyclohexylamine), optionally substituted benzylamines (e.g. phenylbenzylamine and para-bromobenzylamine), ethanolamine, diethanolamine, N-methylglucosamine, N-methylpiperidine, pyridine, substituted pyridines (e.g. collidine, lutidine and 4-dimethylaminopyridine), and tri(hydroxymethyl)methylamine salts; and amino acid salts (e.g., lysine or arginine salts).

Another aspect of the invention provides a pharmaceutical composition that comprises a combination of one or more compounds of formulas I or II or a pharmaceutically acceptable salt thereof and a second therapeutic agent or its pharmaceutically acceptable salt. The second therapeutic agent is a compound selected from the group consisting of azoles; polyenes; purine or pyrimidine nucleotide inhibitors; complex carbohydrate antifungal agents, pneumocandin derivatives and echinocandin derivatives; polyoxins; mannan inhibitors; bactericidal/permeability inducing protein products; elongation factor inhibitors; and immunomodulating agents. More particularly, the second therapeutic agent may be selected from azoles such as fluconazole, voriconazole, intraconazole, ketoconazole, miconazole, ER 30346, and SCH 56592; polyenes such as amphotericin B, nystatin, and liposomal and lipid forms thereof, such as Abelcet™, AmBisome™ and Amphocil™; purine or pyrimidine nucleotide inhibitors such as flucytosine; complex carbohydrate antifungal agents, pneumocandin derivatives or echinocandin derivatives such as caspofungin, enfumafungin, micofungin, and analogs thereof; polyoxins such as nikkomycins, such as nikkomycin Z, and other chitin inhibitors; mannan inhibitors such as predamycin; bactericidal permeability inducing (BPI) protein products such as XMP.97 and XMP.127; and elongation factor inhibitors such as sordarin and analogs thereof. In some embodiments, the second therapeutic agent is a compound selected from the group consisting of intraconazole, flucytosine, fluconazole, amphotericin B and caspofungin.

Yet another aspect of the invention provides an agrochemical composition that comprises one or more compounds of formulas I or II or a salt thereof and an agriculturally acceptable carrier.

Yet another aspect of the invention provides an agrochemical composition that comprises one or more compounds of formulas I or II or an agriculturally acceptable salt thereof and a second active ingredient. The second active ingredient may be selected from the group consisting of herbicides, insecticides, bactericides, nematocides, molluscicides, growth regulators, micronutrients, fertilizers and fungicides.

The compounds of formulas I or II and pharmaceutically or agriculturally acceptable salts thereof, also called “active ingredients” herein, are most effectively utilized when formulated into compositions or formulations with a pharmaceutically or agriculturally acceptable carrier, according to conventional pharmaceutical or agricultural compounding techniques. The term “composition,” as in “pharmaceutical composition” or “agrochemical composition,” is intended to encompass products that comprise one or more active ingredient(s) and inert ingredient(s) that make up the carrier. The term “composition” is also intended to encompass any products which result, directly or indirectly, from combination, complexation, aggregation or other interactions of any two or more active ingredient(s) and/or inert ingredient(s); any products that result, directly or indirectly, from the dissociation of one or more of the active ingredient(s) and/or inert ingredient(s); and any products that result from any other types of reactions of one or more of the active ingredient(s) and/or inert ingredient(s).

The pharmaceutical and agrochemical compositions contain at least a therapeutically effective antifungal amount of active ingredient(s). A “therapeutically effective amount” as used herein refers to an amount of an active ingredient sufficient to produce a desired therapeutic effect. For example, a therapeutically effective antifungal amount of a compound is an amount sufficient to demonstrate antifungal activity and/or inhibit growth of one or more fungal strains.

Therapeutically effective antifungal amounts of active ingredient(s) in pharmaceutical compositions may be provided in a range of about 0.001 mg of active ingredient(s) per kg of patient body weight to about 400 mg active ingredient(s) per kg of patient body weight.

The pharmaceutical compositions and/or agrochemical compositions may be prepared by intimately mixing one or more active ingredient(s) with a carrier, and the components of the carrier may be selected to provide the desired medium. For example, a formulated cream or lotion may be provided by mixing active ingredient(s) into appropriately selected cream or lotion components to provide an active ingredient(s) concentration of between about 0.01% and about 8%.

Pharmaceutical compositions and agrochemical compositions according to aspects of the invention may be formulated as compositions suitable for oral, topical, parenteral (including intraperitoneal (I.P.), subcutaneous, intramuscular and intravenous (I.V.)), nasal and suppository administration, or for administration by insufflation.

When the active ingredient(s) are used as an antifungal agent(s), any suitable method of administration may be used. When the active ingredient(s) are used for treatment of mycotic infections, oral or intravenous administration is usually employed.

For oral administration, pharmaceutical compositions and agrochemical compositions of embodiments may be formulated as liquid or solid compositions. Liquid compositions may be prepared by combining the active ingredient(s) with pharmaceutically or agriculturally acceptable liquid carrier(s), such as water, glycols, oils, alcohols and the like. For solid compositions, the active ingredient(s) may be combined with pharmaceutically or agriculturally acceptable solid carrier(s), such as starches, sugars, kaolin, ethyl cellulose, calcium carbonate, sodium carbonate, calcium phosphate, talc and lactose. These solid carrier(s) may optionally be combined with a lubricant, such as calcium stearate, and/or with a binder-disintegrating agent or the like. Because tablets and capsules are easily administered, these dosage forms may represent the most advantageous oral-dosage form for some situations. Compositions in unit-dosage form also constitute an aspect of the invention.

For administration by injection, pharmaceutical compositions and/or agrochemical compositions of embodiments may be formulated as suspensions, solutions or emulsions. The pharmaceutically or agriculturally acceptable carriers for injectable compositions may be oily vehicles or aqueous vehicles, such as 0.85% sodium chloride in water or 5% dextrose in water. In addition, injectable compositions may include formulating agents, such as buffering agents, solubilizing agents, suspending agents and/or dispersing agents. Buffering agents, as well as additives such as saline or glucose, may be added to make the solutions isotonic. For drip-intravenous administration, the active ingredient(s) may be solubilized in alcohol/propylene glycol or polyethylene glycol. Injectable compositions may be provided as liquid compositions, in unit-dosage form in ampoules or in multidose containers, optionally containing an added preservative. Alternatively, the active ingredient(s) may be provided in powder form, and may be reconstituted in a suitable liquid vehicle prior to administration.

The term “unit-dosage form,” as used in the specification and claims, refers to physically discrete units, each containing a predetermined quantity of active ingredient(s), calculated to produce a desired therapeutic effect, in association with an acceptable carrier. Examples of such unit-dosage forms include tablets, capsules, pills, powder packets, wafers, measured units in ampoules or in multidose containers, and the like.

Yet another aspect of the present invention provides a method for the treatment or prevention of fungal infections, which may be systemic and/or nonsystemic fungal infections, in mammals, including humans and animals, in which the methods comprise administering to the mammal therapeutically effective amounts of active ingredient(s). Methods of this aspect may be used to treat or prevent infection by fungi such as Cryptococcus neoformans, Candida albicans, Candida albicans, Candida glabrata, Candida lusitaniae, Candida parapsilosis, Candida krusei, Candida tropicalis, Saccharomyces cerevisiae, and Aspergillus fumigatus.

Yet another aspect of the present invention provides a method for treatment or prevention of fungal infections in mammals, which comprises administering to said mammal therapeutically effective amounts of active ingredient(s) and a second therapeutic agent selected from the group consisting of azoles; polyenes; purine or pyrimidine nucleotide inhibitors; complex carbohydrate antifungal agents, pneumocandin derivatives and echinocandin derivatives; polyoxins; mannan inhibitors; bactericidal/permeability inducing protein products; elongation factor inhibitors; and immunomodulating agents.

Yet another aspect of the present invention provides a method for controlling phytopathogenic fungi, which comprises administering to a plant in need of such control therapeutically effective amounts of active ingredient(s).

A further aspect of the present invention provides a method for controlling phytopathogenic fungi, which comprises administering to a plant in need of such control therapeutically effective amounts of active ingredient(s) and a second active agent selected from the group consisting of herbicides, insecticides, bactericides, nematocides, molluscicides, growth regulators, micronutrients, fertilizers and fungicides.

As used herein, unless otherwise noted, the following terms have the indicated meanings.

The term “plants” as used herein is intended to include live plants and plant material such as foliage, flowers, seeds, fruits, and other materials derived from plants. The term also includes roots of plants where active ingredient(s) are administered via application to the soil.

The term “mammal” as used herein is intended to include humans, and warm-blooded animals, including domesticated animals such as cats, dogs, livestock, and the like.

Compounds described herein may be prepared by fermentation of fungal strains MF7022 and/or MF7023, and solvent extraction. In embodiments, compounds obtained by fermentation of fungal strains and solvent extraction may be further synthetically modified to yield additional compounds of the invention. Additionally, compounds of the invention may be prepared synthetically.

Fungal strains MF7022 and MF7023 have been identified as Cosmospora sp. (Order Hypocreales), which were isolated from a lichen thallus collected in Miraflores de la Sierra, Madrid, Spain. The fungal strains MF7022 and MF7023 have been deposited under the Budapest Treaty, in the culture collection of the American Type Culture Collection at 10801 University Boulevard, Manassas, Va. 20110-2209 on Sep. 26, 2006, and were assigned accession numbers ATCC No. PAT-7894 and ATCC No. PAT-7895, respectively.

As used herein, the term “biologically pure sample” of a fungal strain refers to a sample of the fungal strain of interest that is provided in a form not found in nature; that is, a biologically pure sample of a fungal strain contains the fungal strain of interest but is substantially lacking in fungal strains, fungal materials and/or other biological materials.

The fungal colonies of strains MF7022 and MF7023 exhibit the following morphological features in different agar media when grown under continuous fluorescent light for 21 days at 22° C. On 2% malt extract (Difco™) agar, colonies attained 30-31 mm diameters, the mycelium was submerged to apprised, with scant velvety mycelium at the center; colony margins were even, and appeared hyaline at the edge; colony colors were pale yellow to golden yellow or yellowish brown, with the reverse same. On cornmeal (Difco™) agar, colonies attained 40-45 mm diameters, the mycelium were submerged and hyaline in appearance, and colony margins were minutely fimbriate. On oatmeal (Difco™) agar, colonies attained 39-40 mm diameters, the mycelium was appressed to velvety and hispid at the center; colony colors were pale yellow to golden with white aerial mycelium, becoming subzonate, with some sectoring and sectors lacking aerial mycelium; colonies were dark golden in reverse.

A conidial stage was only observed on cornmeal agar at relatively low temperatures (16° C.) after prolonged incubation (>6 weeks). Scattered sporodochia formed at the colony edges when mycelium completely covered the agar surface and ceased to extend. Sporodochia were hyaline to pale orange or pinkish orange and up to 300 μm in diameter, consisting of one to several conidiophores that arose from the agar surface. Conidiophores consisted of short parallel branches arising from surface hyphae that gave rise to sparse or dense arrangements of penicillately branched conidiogenous cells, with primary branches septate or not, consisting of cylindrical to clavate cells, up to 7.5 μm in diameter, branching 2 to 4 times, and terminating in conidiogenous cells. Conidiogenous cells were phialidic, enteroblastic, hyaline, 7-15 μm long, 2-3 μm wide, cylindrical and tapered at apex, often with an inconspicuous collerette at conidiogenous locus. Conidia were strongly curved to lunate, hyaline, overwhelmingly 3-septate, occasionally with slightly truncated or flattened basal cells, 15-20 μm long and 4-6 μm wide. The conidial state was very similar to the conidial state of Cosmospora auranticola, which has been referred to in the literature as Fusarium larvum, a fungus generally associated with scale insects and white flies (C. Booth, The Genus Fusarium (Commonwealth Mycological Institute, Kew, United Kingdom 1971); Amy Y. Rossman et al., Genera of Bionectriaceae, Hypocreaceae and Nectriaceae (Hypocreales Ascomycetes), in 42 Studies in Mycology (1999)).

Sequences of ribosomal DNA (rDNA) genes of fungal strains MF7022 (ATCC No. PAT-7894) and MF7023 (ATCC No. PAT-7895) were determined to aid in identification of other fungi and to interfere with phylogenetic relationships of the strains to other fungi. The DNA of strains MF7022 and MF7023 were extracted and used as templates for PCR reactions, following standard molecular biology procedures. A DNA fragment of 28S rDNA, containing domains D1 and D2 of this gene, was amplified and sequenced. Sequences from the two strains were identical, indicating that they were conspecific and possibly clones from the same population. The sequences obtained were used to query the GenBank database for similar ribosomal sequences. The best matches retrieved, with percentage similarities observed, were: Cosmospora flammea (U88103) 97%, Fusarium sp. (NRRL25126, AF228357) 96%, Fusarium sp. (NRRL26790 AF228359) 98%, Fusarium sp. (NRRL26803, AF228360) 98%, and Fusarium larvarum (NRRL20473, U88107) 100%. The fungi that consistently exhibited high sequence similarity all belonged to the order Hypocreales. The results confirmed that fungal strains MF7022 and MF7023 belong to the Hypocreales order and suggest that these strains are congeneric with fungi of the genus Cosmospora.

### Fermentation Procedure

The fungal strains MF7022 (ATCC No. PAT-7894) and MF7023 (ATCC No. PAT-7895), identified as Cosmospora sp., are usually cultured in an aqueous nutrient medium containing sources of assimilable carbon and nitrogen. For example, the cultures may be grown under submerged aerobic conditions (e.g., such as shaking the culture, submerging the culture, etc.). The aqueous medium is preferably maintained at a pH of about 6-8, at the initiation and termination (harvest) of the fermentation process. The desired pH may be maintained by the use of a buffer, such as morpholinoethane-sulfonic acid (MES), morpholinopropane-sulfonic acid (MOPS), and the like, or by choosing nutrient materials that inherently possess buffering properties.

Suitable sources of carbon in the nutrient medium include carbohydrates, such as glucose, xylose, galactose, glycerine, starch, sucrose, dextrin and the like. Other suitable carbon sources that may be used include maltose, rhamnose, raffinose, arabinose, mannose, sodium succinate and the like.

Suitable sources of nitrogen are yeast extracts, meat extracts, peptone, gluten meal, cottonseed meal, soybean meal and other vegetable meals (partially or totally defatted), casein hydrolysates, soybean hydrolysates, yeast hydrolysates, corn steep liquor, dried yeast, wheat germ, feather meal, peanut powder, distiller's solubles and the like, as well as inorganic and organic nitrogen compounds, such as ammonium salts (including ammonium nitrate, ammonium sulfate, ammonium phosphate, etc.), urea, amino acids, and the like.

The carbon and nitrogen sources, which may be advantageously employed in combination, need not be used in their pure forms; because less pure materials, which contain traces of growth factors, vitamins and significant quantities of mineral nutrients, are also suitable for use. When desired, mineral salts, such as sodium or calcium carbonate, sodium or potassium phosphate, sodium or potassium chloride, sodium or potassium iodide, magnesium salts, copper salts, cobalt salts, and the like, may be added to the medium. If necessary, especially when a culture medium foams excessively, one or more defoaming agent(s), such as liquid paraffin, fatty oils, plant oils, mineral oils or silicones, may be added.

Submerged aerobic cultural conditions are typical methods of culturing cells for the production of cells in massive amounts. For small-scale production, a shaking or surface culture in a flask or bottle may be employed. When growth is carried out in large tanks, it may be preferable to use the vegetative forms of the organism for inoculation in the production tanks in order to avoid growth lag in the process of production. Accordingly, it may be desirable first to produce a vegetative inoculum of the organism by inoculating a relatively small quantity of culture medium with spores or mycelia of the organism produced in a “slant” or Petri dish and culturing said inoculated medium, also called the “seed medium,” and then to transfer the cultured vegetative inoculum aseptically to large tanks. The fermentation medium, in which the inoculum is produced, is generally autoclaved to sterilize the medium prior to inoculation. The pH of the medium is generally adjusted to about 6-7 prior to the autoclaving step.

Agitation and aeration of the culture mixture may be accomplished in a variety of ways. Agitation may be provided by a propeller or similar mechanical agitation equipment, by revolving or shaking the fermentor, by various pumping equipment, or by the passage of sterile air through the medium.

The fermentation is usually conducted at a temperature between about 20° C. and 30° C., such as between about 22° C. and 25° C., for a period of about 14-28 days, and parameters may be varied according to fermentation conditions and scales.

Preferred culturing/production media for carrying out the fermentation include the media as set forth in the examples.

### Isolation and Characterization of Compounds

Compounds of the invention, that is compounds of formulas I and II in which R¹ is hydrogen, may be extracted as an interconverting mixture of isomeric compounds from liquid or solid fermentations of either fungal culture by diluting with acetone and mixing for several hours at room temperature. The mixture is then filtered and the filtrate concentrated to a mostly aqueous solution containing compounds of formulas I and II, in which each R¹ is hydrogen. Other suitable extraction solvents include tetrahydrofuran, methanol and ethanol. An immiscible solvent such as methyl ethyl ketone or ethyl acetate is also suitable.

The product from solid or liquid fermentations is recovered from the extract by adsorption/elution on polystyrene-divinyl benzene resins. The eluate is then further purified by liquid-liquid partitioning by high-speed countercurrent chromatography. The product is then further isolated by chromatography, which may, in some embodiments, be conducted on reverse phase silica (including C8 and C18) based resins. The product compounds may be chemically modified to produce alkylated compounds, that is compounds of formulas I and II in which R¹ is alkyl.

A preferred adsorption/elution for the capture of compounds of formulas I and II from crude fermentation extract is with polystyrene-divinyl benzene resins, such as HP20 and SP207 (Mitsubishi). The crude extract containing compounds of formulas I and II is dissolved in a mixture of acetone and water, adjusted to approximately pH 3, and adsorbed onto a bed of resin. Compounds of formulas I and II are adsorbed onto the resin at low organic-solvent concentrations, high aqueous concentrations, and eluted by washing the resin with a solution that is mostly an organic solvent, such as acetonitrile or methanol. High-speed countercurrent chromatography may also be used for the purification of the compounds of formulas I and II from ethyl acetate extracts containing the compounds of formulas I and II.

Preferred methods for final purification of compounds of formulas I and II are high-speed countercurrent chromatography or reverse-phase liquid chromatography. The solvents for high-speed countercurrent chromatography are hexane, ethyl acetate, methanol and water. For reverse-phase chromatography, the stationary phase may be either a C8 or C18 bonded phase. A preferred eluant is a buffered mixture of water and acetonitrile or methanol. After chromatography, the natural product is separated from non-volatile buffer components by adsorption onto a polymeric hydrophobic resin, such as HP20 or SP207, and elution with organic solvent, such as acetonitrile or methanol. Compounds of formulas I and II can then be recovered by concentration in vacuo, filtration after concentration to an aqueous solution, or by lyophilization after removal of the acetonitrile or methanol.

The recovered compounds can then be further characterized by infrared spectroscopy, NMR spectroscopy, and mass spectroscopy.

### Inoculate Seed Culture

The culture was maintained on agar plugs in vials containing sterile 10% glycerol and stored at −80° C. until ready for use. The seed culture was inoculated by aseptically transferring four agar plugs into a 250 mL Erlenmeyer flask containing 60 mL seed medium of the following composition (in g/liter):
Corn steep powder, 2.5
Tomato paste, 40.0;
Oat flour, 10.0;
Glucose, 10.0 and
Trace elements solution, 10 mL/liter.

The Trace elements solution consisted of the following components:
FeSO₄.7H₂O, 1.0 g/liter;
MnSO₄.4H₂O, 1.0 g/liter;
CuCl₂.2H₂O, 0.25 g/liter;
CaCl₂.2H₂O, 0.1 g/liter;
H₃BO₃. 0.056 g/liter;
(NH₄)₆MoO₂₄.4H₂O, 0.019 g/liter;
ZnSO₄.7H₂O, 1.0 g/liter;
dissolved in 0.6 N HCl.

Seed medium was prepared with distilled water. The pH was adjusted to 6.8 by adding sodium hydroxide, and the medium was dispensed into 250 mL Erlenmeyer flasks and capped with cellulose plugs before being autoclaved at 121° C. for 20 minutes.

The seed culture was incubated at 22° C. on a gyratory shaker (220 rpm) for five days prior to the inoculation of fermentation flasks.

### Example 1

### Fermentation of the MF7022

The production medium was prepared with distilled water, and the pH was adjusted to 6.5. A culture of a Cosmospora sp. (Merck Culture Collection MF7022 (ATCC No. PAT-7894)) was grown in a medium containing maltose (75.0 g), V8™ juice (200 mL), soy flour (1.0 g), L-proline (3.0 g), MES buffer (16.2 g), and distilled H₂O (800 mL) for 20 days at 22° C. After harvesting the fermentation (1 L), the whole broth was extracted with one volume of acetone, filtered to remove the mycelium, and concentrated to remove acetone, providing a sample that contains a mixture of compounds of formulas I and II (subsequently referred to as Natural Product I or NPI), as an equilibrating mixture of isomeric forms. The titer of NPI in fermentation broth was typically 1.0-1.2 g/L. After acetone extraction of whole broth, a mixture of isomeric forms was observed by analysis with C18 HPLC.

### Isolation of Natural Product I (NPI)

The extract containing the mixture of isomeric forms was fractionated with CHP20P (MC1) resin (50 mL), eluting with a gradient of acetonitrile in 10 mM potassium phosphate buffer at pH 3. The rich cut of activity (as measured by inhibition of growth of Candida albicans in an agar based assay) was pooled, and a portion of this material was fractionated by countercurrent chromatography (CCC) with an elution/extrusion system of 1:1:1:1 hexane:ethyl acetate:methanol:water (V_{c}=200 mL, upper phase=mobile phase, 1000 rpm, 10 mL/min). The active fractions from the CCC fractionation were analyzed by analytical HPLC (Waters Symmetry300, 300 Å, C18, 4.6×50 mm, 5 μm, 0-70% ACN in 1:9 ACN:10 mM potassium phosphate pH 3, 2 mL/min, 25° C.) and identified a mixture of related components that were responsible for antifungal activity. Further chromatography (C18 HPLC) on this sample separated these components, but equilibration over 10-20 hours (room temperature) returned the purified components back to the original mixture of isomeric forms. The equilibrium mixture contained two major components and two minor components as analyzed by HPLC and NMR. Analysis by mass spectrometry of these samples indicated that these components all had identical molecular weights that were consistent with a molecular formula of C₂₃H₁₇NO₉.

### Structure Elucidation

In order to elucidate the structure of the natural product, freshly prepared ethereal diazomethane was added to a stirred mixture of NPI suspended in 1:1 methanol:diethyl ether at −78° C. The reaction progress was monitored by HPLC. When the analysis indicated that the starting mixture of components was consumed, acetic acid (1 mL) was added to quench excess diazomethane and the reaction was concentrated in vacuo. Preparative C18 HPLC (Waters Symmetry C18 300 Å, 19×300 mm, 7 μm) was used to separate and purify the methylated components from the crude reaction mixture. As determined by mass spectroscopy, each methylated component contained a single additional methyl group as compared with the starting natural product. The methylated analogs of compounds A and B co-eluted in the above chromatography. These compounds could be separated from the methylated analogs of compounds C and D.

The mono-methylated analogs of Compounds A and B were crystallized from 1:1 CH₂Cl₂:methanol and an x-ray structure was obtained to confirm the structure of the natural product. The x-ray structure was a 1:1 co-crystal of methylated compounds A and B. The structures of the methylated analogs of Compounds C and D were confirmed by NMR spectroscopy. NMR analysis of the methylated products and the mixture of unmodified natural products fully supported the structure determined by x-ray analysis. NMR data on the natural product was assigned for each of the major and minor isomers, which exist as a mixture in a single sample. Relative stereochemistry is shown below:

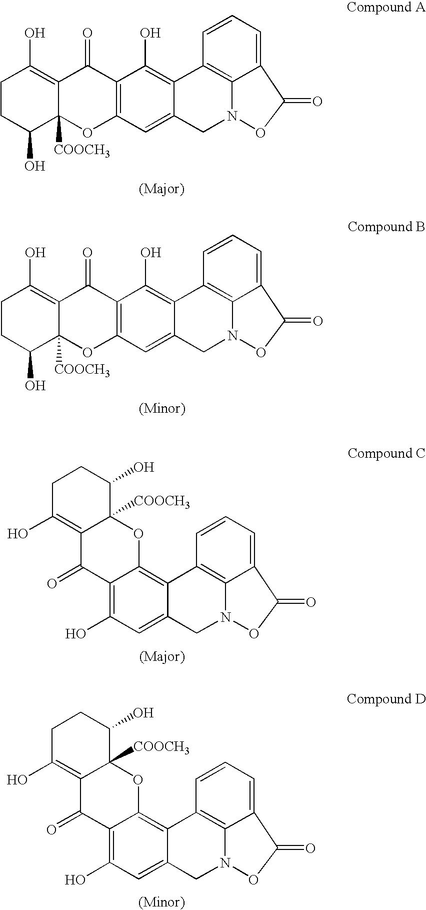

### Compound A (Major Diastereomer (Syn))

¹³C NMR (126 MHZ, DMSO): δ 25.3, 27.7, 53.0, 54.8, 70.0, 85.3, 101.1, 106.5, 108.5, 109.8, 109.9, 118.7, 122.8, 125.9, 130.7, 142.0, 155.5, 159.5, 160.9, 167.2, 169.7, 179.8, 185.6;

¹H NMR (499 MHZ, DMSO): δ 1.91 (M, 1H), 2.13 (M, 1H), 2.57 (M, 1H), 2.82 (M, 1H), 3.56 (S, 3H), 4.20 (DT, 1H), 4.67 (D, 1H), 4.70 (D, 1H), 5.95 (D, 1H), 6.72 (S, 1H), 7.34 (T, 1H), 7.65 (D, 1H), 8.27 (D, 1H), 12.35 (S, 1H).

### Compound B (Minor Diastereomer (Anti))

¹³C NMR (126 MHz, DMSO): δ 23.8, 24.7, 53.6, 54.7, 65.7, 84.5, 100.6, 106.6, 108.5, 109.9, 110.4, 118.7, 122.9, 125.9, 130.8, 142.0, 155.7, 158.2, 158.8, 167.2, 171.1, 180.9, 185.5;

¹H NMR (499 MHz, DMSO): δ 1.82 (m, 1H), 1.95 (m, 1H), 2.45 (m, 1H), 2.74 (m, 1H), 3.59 (s, 3H), 4.39 (m, 1H), 4.68 (ob, 1H), 4.70 (ob, 1H), 5.97 (ob, 1H), 6.74 (s, 1H), 7.40 (t, 1H), 7.67 (d, 1H), 8.32 (d, 1H), 11.62 (s, 1H).

### Compound C (Major Diastereomer (Syn))

¹³C NMR (126 MHz, DMSO): δ 25.4, 28.0, 52.9, 55.0, 70.2, 85.5, 101.5, 106.9, 109.2, 109.7, 110.4, 118.7, 122.7, 126.0, 132.2, 141.8, 155.6, 156.9, 161.0, 167.3, 169.5, 180.5, 184.6;

¹H NMR (499 MHz, DMSO): δ 1.97 (m, 1H), 2.17 (m, 1H), 2.60 (m, 1H), 2.85 (m, 1H), 3.59 (s, 3H), 4.30 (dt, 1H), 4.55 (d, 1H), 4.76 (d, 1H), 6.01 (d, 1H), 6.67 (s, 1H), 7.32 (t, 1H), 7.66 (d, 1H), 8.67 (d, 1H), 11.47 (s, 1H).

### Compound D (Minor Diastereomer (Anti))

¹³C NMR (126 MHz, DMSO): δ 24.0, 24.9, 53.6, 54.9, 65.3, 84.9, 100.8, 106.2, 109.2, 109.8, 110.9, 118.6, 123.0, 126.0, 132.0, 142.0, 155.5, 158.9, 158.9, 167.2, 170.9, 181.9, 185.9;

¹H NMR (499 MHz, DMSO): δ 1.83 (m, 1H), 1.97 (m, 1H), 2.43 (m, 1H), 2.73 (m, 1H), 3.65 (s, 3H), 4.24 (m, 1H), 4.62 (d, 1H), 4.68 (d, 1H), 5.89 (d, 1H), 6.72 (s, 1H), 7.36 (t, 1H), 7.70 (d, 1H), 8.63 (d, 1H), 12.48 (s, 1H).

ESI-FTMS C₂₃H₁₇NO₉; [M+H]⁺ exp 452.0976, calc 452.0982.

UV (acetonitrile): λₘₐₓ 343 (ε=29,400 M⁻¹ cm⁻¹), 290 (ε=15,100 M⁻¹ cm⁻¹), 265 (ε=16,200 M⁻¹ cm⁻¹), 234 (ε=28,800 M⁻¹ cm⁻¹).

IR (thin film): 3475, 1750, 1611, 1579, 1500, 1455, 1431, 1364, 1348 cm⁻¹.

[α]_{D} 38.0° (c=0.2, CH₂Cl₂).

### Methylated Compound A

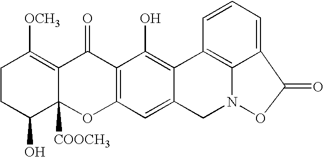

¹³C NMR (126 MHz, DMSO): δ 24.0, 25.3, 52.7, 54.8, 56.2, 69.8, 87.0, 103.3, 107.4, 107.5, 109.4, 109.8, 119.1, 122.6, 125.9, 130.5, 141.1, 155.5, 159.3, 160.1, 167.3, 169.8, 173.2, 184.8;

¹H NMR (499 MHz, DMSO): δ 1.87 (m, 1H), 2.06 (m, 1H), 2.83 (m, 1H), 2.83 (m, 1H), 3.55 (s, 3H), 3.88 (s, 3H), 4.15 (m, 1H), 4.70 (s, 1H), 5.92 (d, 1H), 6.68 (s, 1H), 7.36 (t, 1H), 7.66 (d, 1H), 8.34 (d, 1H), 14.13 (s, 1H).

ESI-FTMS C₂₄H₁₉NO₉; exp 466.1128, calc 466.1138.

### Methylated Compound C

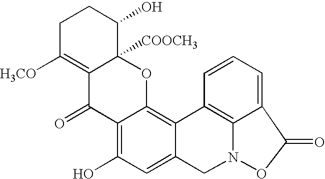

¹³C NMR (126 MHz, DMSO): δ 24.6, 25.5, 53.0, 55.0, 56.0, 70.0, 87.4, 103.0, 107.7, 108.3, 109.6, 109.9, 119.1, 122.3, 125.8, 131.8, 141.0, 155.6, 156.6, 162.4, 167.4, 169.6, 173.1, 184.2;

¹H NMR (499 MHz, DMSO): δ 2.00 (m, 1H), 2.17 (m, 1H), 2.88 (m, 2H), 3.55 (s, 3H), 3.90 (s, 3H), 4.30 (dt, 1H), 4.55 (d, 1H), 4.80 (d, 1H), 5.97 (d, 1H), 6.60 (s, 1H), 7.36 (t, 1H), 7.70 (d, 1H), 8.68 (d, 1H), 13.25 (s, 1H).

ESI-FTMS C₂₄H₁₉NO₉; [M+H]⁺ exp 466.1129, calc 466.1138.

### In Vitro Assay—Susceptibility Testing

### Drug Stock Solutions and Dilutions

To each well of a 96-well plate (with columns 1-12 and rows A-H), 100 μL of appropriate test medium (example: RPMI-1640 containing 0.165 molar MOPS+3 g/L glutamine w/o sodium bicarbonate or RPMI-1640 containing 0.165 molar MOPS+3 g/L glutamine w/o sodium bicarbonate with 3.2% DMSO or 2×RPMI-1640 containing 0.33 molar MOPS+6 g/L glutamine w/o sodium bicarbonate with 6.4% DMSO for the plates with final concentration of 50% serum or Cation Adjusted Muller Hilton Broth (CAMHB) with 3.2% DMSO) was added using the Thermal-LabSystems MULTIDROP™ dispenser. The Clinical Laboratory Standards Institute (CLSI) (formerly National Committee for Clinical Laboratory Standards (NCCLS)) formula was used to calculate the amount of dilution needed for a standard solution. The test compound was dissolved at concentration of 10 mg/mL in DMSO and diluted 1:78 into appropriate test medium with no DMSO or 1.92% DMSO or 5.12% DMSO. The drug concentration achieved was 128 μg/ml and DMSO concentration of 3.2%.

To the first well of each row, 100 μL of the compound stock solutions (128 μg/mL) were added with multichannel Matrix pipette. Compounds were serially diluted two-fold with Perkin Elmer CETUS PRO/PETTE™ diluter or TECAN™ (100 μL taken from first well of each row and placed into second well and mixed, 100 μL of second well of each row taken and placed into third well and mixed, etc.) across the plate to column 11 (column 12 was the growth control well-no drug), and the last 100 μL was discarded, yielding compound concentrations of 64 to 0.06 μg/mL. For plates with dermatophytes, the last 100 μL were placed in the first row of a second plate and serial diluted two-fold and yielding compound concentrations of 64-0.00004 μg/mL. Amphotericin B and caspofungin acetate (CANCIDAS™), the control compounds, were prepared as a stock solution of 10 mg/mL in DMSO and prepared in micro-titer plate as stated above for test compounds.

### Yeast Inoculation

In the microbroth dilution assay for yeasts, microorganisms Candida spp., Cryptococcus neoformans (MY2062) and Saccharomyces cerevisiae (MY2255) were selected by streaking a yeast culture on Sabouraud Dextrose Agar (SDA), incubating for 24-48 hours at 35-37° C., thereafter selecting one characteristic colony and transferring to a fresh plate and incubating under same conditions. From the regrowth, 3 to 5 colonies were selected and suspended in 5 mL of sterile Normal saline (BBL) and adjusted to match the turbidity of a 0.5 McFarland standard using Dade/Behring turbidity meter (preferred OD of 0.06 to 0.12). This resulted in a concentration of approximately 1−5×10⁶ colony forming units per milliliter (CFU/mL). The inocula were further diluted 1:50 into RPMI-1640 containing 0.165 molar MOPS+3 g/L glutamine w/o sodium bicarbonate with 3.2% DMSO (0.1 mL into 4.9 mL) and further diluted 1:20 in the same medium (3.2 ml of 1:50 dilution+60.8 ml RPMI-1640 containing 0.165 molar MOPS+3 g/L glutamine w/o sodium bicarbonate with 3.2% DMSO). Assay plates previously titrated with drug in RPMI-1640 containing 0.165 molar MOPS+3 g/L glutamine w/o sodium bicarbonate with 3.2% DMSO were then inoculated with 100 μL per well of this dilution of culture. This results in a final organism concentration of 5×10² to 2.5×10³ CFU/mL and final compound concentrations of 32 to 0.03 mg/mL. In addition, C. albicans (MY1055) was also tested with heat inactivated (1 hour at 55° C.) mouse serum which was filtered twice using 0.22 micron GP Express PLUS Millipore filtration system. This standardized suspension was diluted 1:50 into mouse serum (0.1 mL into 4.9 mL) and further diluted 1:20 in mouse serum (3.2 mL of 1:50 dilution into 60.8 mL mouse serum). Assay plates previously titrated with drug in 2×RPMI-1640 containing 0.33 molar MOPS+6 g/L glutamine w/o sodium bicarbonate with 6.4% DMSO were then inoculated with 100 μL per well of this dilution of culture. This results in a final organism concentration of 5×10² to 2.5×10³ CFU/mL and final compound concentration of 32 to 0.03 μg/mL and 50% mouse serum. Plates were incubated at 35-37° C. and MICs were read at 24 hours for Candida and 48 hours for Cryptococcus neoformans. Viable cell counts were performed on 0.5 McFarland samples to verify the CFU/mL. A 1:100 dilution was made in sterile Normal saline with the 0.5 McFarland (0.1 mL+9.9 mL saline). This was followed by three ten-fold dilutions (0.5 mL+4.5 mL saline). 100 μL of each dilution (10⁴, 10⁵, 10⁶) was spread onto a Sabouraud Dextrose Agar (SDA) plates which were then incubated for 24 to 48 hours at 35° C. After incubation colonies were counted and recorded, growth and sterility controls for each organism were also carried out. Column 12 was the growth control and contains no drug. Row H was not inoculated with organism or drug and was used as sterility control.

The minimum inhibitory concentration (MIC-100) for all compounds is determined to be the lowest concentration of compound at which there was no visible growth as compared to growth control without drug. The minimum prominent inhibition (MIC-80) in growth was indicated as 80% inhibition in growth compared to growth control without drug. For Aspergillus and dermatophyte T. mentagrophytes minimum effective concentration (MEC) (narly morphology of hyphae both macroscopic and microscopic) was recorded. The MIC for all Candida species were recorded at 24 hours incubation, the bacteria were recorded at 20 hours, Cryptococcus and Aspergillus were recorded at 48 hours and dermatophyte were recorded at 96 hours.

Natural Product I (as isolated in Example 1) was evaluated for antifungal activity. MIC was observed for Natural Product I against the following strains:

TABLE I

| Strain | MIC-100 (MIC-80) (μg/ml) | |
|---|---|---|
| Candida albicans | 0.125 (<0.03) | μg/mL |
| Candida albicans | 4 (0.5) | μg/mL |
| (in the presence of 50% mouse serum) | | |
| Candida glabrata | 32 (16-32) | μg/mL |
| Candida lusitaniae | 4 (0.25-0.5) | μg/mL |
| Candida parapsilosis | 8-16 (2-4) | μg/mL |
| Candida krusei | 0.125-0.25 (<0.03) | μg/mL |
| Candida tropicalis | 2 (1) | μg/mL |
| Saccharomyces cerevisiae | 32 (16) | μg/mL |

### Example 2

### In Vitro Assay

### Activity Against Aspergillus in Modified Media

Sabouraud-dextrose 1.8% agar (12 mL) was equilibrated at 55° C., seeded with Aspergillus fumigatus MF5668 spores at 1−5×10⁶/mL, added to a 12×8 cm Omnidish and allowed to solidify. A solution of Natural Product I (2 μL, 1-2 mg/ml in DMSO) was applied to the agar surface, and the plate was incubated at 37° C. for 16 hours. Inhibitory activity of Natural Product I against A. fumigatus was observed.

### Example 3

### Target Organ Kidney Assay (TOKA)

### Evaluation of In Vivo Efficacy Against Candida albicans

DBA/2 female mice (Taconic) weighing 20 grams were used. This inbred strain of mouse is congenitally immune deficient in Complement Component 5. Candida albicans MY1055 (Merck Culture Collection), a human clinical pathogenic isolate originally obtained from Williamsburg Community Hospital, Williamsburg, Va., (MCV #7.270) was used. Growth from an overnight Sabouraud-dextrose agar (SDA) culture was suspended in sterile saline and cell concentrations were determined by hemacytometer count. The cell suspension of C. albicans MY1055 was adjusted to 1.50×10⁵ cells/mL by dilution in sterile physiological saline. When 0.2 mL of this cell suspension was administered by I.V. in the tail veins of mice, the final inoculum was 3.0×10⁴ cells/mouse (approximately one 14 day LD₅₀).

Therapy was initiated within 15 minutes after challenge and mice were treated for a total of two days. Natural Product I was administered I.P., b.i.d. at test doses of 50, 25, and 12.5 mg/kg. The target organ assay for Candida species monitors the number of colony forming units (CFU) per gram of paired kidneys at time points following challenge (target organ kidney assay (TOKA)). Paired kidneys from euthanized mice (4/group) were removed using aseptic technique, weighed and placed in sterile Whirl Pak bags (Fisher Scientific) containing 5 mL sterile saline. Kidneys were homogenized in the bags, serially diluted in saline and aliquots were plated on SDA. Plates were incubated at 35° C., and the organisms were enumerated after 48 hours. The mean log₁₀ CFU per gram in the kidneys of the treated groups was compared with those in the kidneys of sham-treated mice.

For Natural Product I, reductions from sham were observed at 50 mpk (2.30, 2.88 log), 25 mpk (2.11, 1.86 log) and 12.5 mpk (1.00, 1.72 log).

What is claimed is:

1. A purified composition comprising one or more compounds selected from the group of compounds of formulas I and II and pharmaceutically or agriculturally acceptable salts thereof: 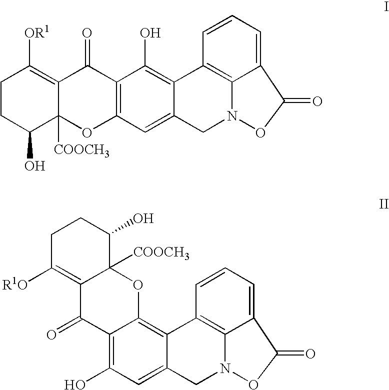
wherein R¹ is selected from the group consisting of hydrogen and C₁-C₆ alkyl groups.

2. The composition according to claim 1, wherein R¹ is hydrogen.

3. The composition according to claim 2, wherein the composition is a mixture of naturally occurring compounds of formulas I and II and pharmaceutically or agriculturally acceptable salts thereof.

4. A compound selected from the group of compounds of formulas I and II and pharmaceutically or agriculturally acceptable salts thereof: 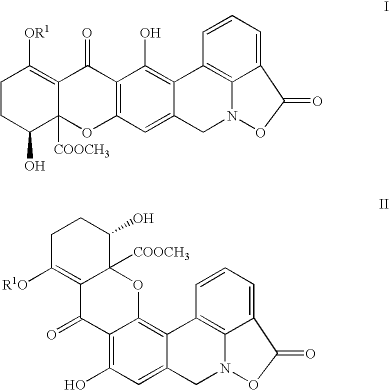
wherein R¹ is selected from the group consisting of hydrogen and C₁-C₆ alkyl groups.

5. The compound according to claim 4, wherein R¹ is hydrogen.

6. A pharmaceutical composition comprising one or more compounds according to claim 4 and a pharmaceutically acceptable carrier.

7. A pharmaceutical composition comprising a combination of one or more compounds according to claim 4 and a second therapeutic agent or a pharmaceutically acceptable salt of the second therapeutic agent.

8. The pharmaceutical composition according to claim 7, wherein the second therapeutic agent is a compound selected from the group consisting of azoles; polyenes; purine or pyrimidine nucleotide inhibitors; complex carbohydrate antifungal agents, pneumocandin derivatives and echinocandin derivatives; polyoxins; mannan inhibitors; bactericidal/permeability inducing protein products; elongation factor inhibitors; and immunomodulating agents.

9. The pharmaceutical composition according to claim 7, wherein the second therapeutic agent is a compound selected from the group consisting of intraconazole, flucytosine, fluconazole, amphotericin B, and caspofungin.

10. An agrochemical composition comprising one or more compounds according to claim 4 and an agriculturally acceptable carrier.

11. An agrochemical composition comprising one or more compounds according to claim 4 and a second active ingredient selected from the group consisting of herbicides, insecticides, bactericides, nematocides, molluscicides, growth regulators, micronutrients, fertilizers and fungicides.

12. A method of treating or preventing fungal infection in a mammal, the method comprising administering to the mammal therapeutically effective amounts of one or more compounds according to claim 4.

13. A method of treating or preventing fungal infection in a mammal, the method comprising administering to the mammal therapeutically effective amounts of one or more compounds according to claim 4 and a second therapeutic agent or a pharmaceutically acceptable salt of the second therapeutic agent;
wherein the second therapeutic agent is a compound selected from the group consisting of azoles, polyenes, purine pyrimidine nucleotide inhibitors, pneumocandin derivatives, echinocandin derivatives, polyoxins, mannan inhibitors, bactericidal/permeability inducing protein products, elongation factor inhibitors, and immunomodulating agents.

14. A method for controlling phytopathogenic fungi, the method comprising administering to a plant in need of such control therapeutically effective amounts of one or more compounds according to claim 4.

15. A method for controlling phytopathogenic fungi, the method comprising administering to a plant in need of such control therapeutically effective amounts of one or more compounds according to claim 4 and a second active ingredient selected from the group consisting of herbicides, insecticides, bactericides, nematocides, molluscicides, growth regulators, micronutrients, fertilizers and fungicides.

16. A biologically pure culture of Cosmospora sp. (Order Hypocreales) deposited with the American Type Culture Collection as ATCC No. PAT-7894.

17. A process of preparing the composition according to claim 2, comprising culturing and fermenting a culture of Cosmospora sp. ATCC No. PAT-7894.

18. A biologically pure culture of Cosmospora sp. (Order Hypocreales) deposited with the American Type Culture Collection as ATCC No. PAT-7895.

19. A process of preparing the composition according to claim 2, comprising culturing and fermenting a culture of Cosmospora sp. ATCC No. PAT-7895.
